(19) United States
(12) Patent Application Publication
(10) Pub. No.: US 2024/0133881 A1
(43) Pub. Date: Apr. 25, 2024

(54) DEVICES AND KITS FOR DETECTING ANALYTES OF INTEREST AND METHODS OF USING THE SAME

(71) Applicant: Quidel Corporation, San Diego, CA (US)

(72) Inventors: Valerio Pini, Tres Cantos (ES); Adonis Stassinopoulos, Del Mar, CA (US); Matthias Mösl, Anif (AT); Jesús Óscar Ahumada Heredero, Tres Cantos (ES); Chloé Alexia Rodriguez, Tres Cantos (ES); Andreas Thon, Tres Cantos (ES); Houman Yaghoubi, San Diego, CA (US); Scott Rongey, San Diego, CA (US)

(21) Appl. No.: 18/491,674

(22) Filed: Oct. 19, 2023

Related U.S. Application Data
(60) Provisional application No. 63/417,965, filed on Oct. 20, 2022.

(51) Int. Cl. G01N 33/558; B82Y 30/00

(52) U.S. Cl. CPC G01N 33/558; B82Y 30/00

(57) ABSTRACT

Disclosed are various embodiments of a device comprising a synthetic polymeric substrate having a high quality finish upper surface, the upper surface having at least a bilayer coating comprising a first, reflective layer and a second, transparent layer. Also disclosed are kits containing embodiments of the disclosed device and detectable particles. Also disclosed are various embodiments of a method of using the disclosed device and various embodiments of a method of using the disclosed kit.

### CROSS-REFERENCE TO RELATED APPLICATIONS

This application claims the benefit of U.S. Provisional Application No. 63/417,965, filed Oct. 20, 2022, which is incorporated by reference herein its entirety.

### TECHNICAL FIELD

The subject matter described herein relates composite, solid supports for use in bioassays for determining the presence of one or more analytes of interest. The subject matter described herein further relates to kits comprising said composite, solid supports, as well as methods of using said composite, solid supports and said kits.

### BACKGROUND

Bioassays are used to probe for the presence and/or the quantity of an analyte material in a biological sample. In surface-based assays, the analyte species is captured and detected on a solid support or substrate. Examples of surface-based assays include a DNA or RNA microarray, used for the study of gene expression and genotyping, and arrays with one or more binding moieties such as carbohydrates, antibodies, proteins, haptens or aptamers.

Bioassays typically capture and immobilize a sufficient amount of an analyte from a test sample to provide a detectable signal when interrogated, for example, optically (e,g., using optical tags such as fluorophores, plasmonic nanoparticles, plasmonic substrates, and the like). To be gainfully applied to profiling experiments, the solid support for the bioassay generally must have a highly reproducible surface in terms of surface finish (roughness), optical properties, and/or mechanical properties (e.g., thickness, dimensions, positioning). Having a highly reproducible substrate surface is particularly desirable in assay formats in which the sample and the control must be analyzed on disparate support surfaces with which they are associated, e.g., different supports or different locations on the same support. Supports that are not highly reproducible can result in significant errors when the assay is performed, due to variations from support to support or different locations on the same support.

The present disclosure provides improved solid supports for use in bioassays.

In an aspect, the present disclosure provides various embodiments of a polymer-based device having one or more deposited metal and/or dielectric layers. The disclosed embodiments represent an improvement over expensive ultra-flat silicon-based chips (e.g., ultra-flat supports made from crystalline silicon), which require more complicated manufacturing methods.

Additionally, embodiments comprising deposited metal and/or dielectric layers have been found to allow detection of individual particles in a bioassay. Devices disclosed herein comprising two or more layers achieve substantially improved signal-to-noise ratio (SNR) for individual particles in a bioassay using an optical instrument, such as a dark-field optical microscope or dark-field spectrophotometer.

In certain embodiments described herein, a chemical or biological coating may be applied. Persons of ordinary skill in the art will recognize how to configure the coated device to be compatible with a particular selected chemical coating. In some embodiments, a chemical overcoating is applied to the one or more deposited metal and/or dielectric layers. In some embodiments, a chemical overcoating is applied to a multilayer coating made of metal and/or dielectric layers.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a device is provided that comprises a synthetic polymeric substrate having an upper surface, the upper surface having a coating which comprises a first layer comprised of a material that reflects electromagnetic radiation and a second layer comprised of a material that is dielectric and transparent. In an aspect, the ultra-high quality surface roughness is provided on the upper surface in the device. In certain embodiments, the surface roughness of the upper surface has a surface finish quality which is comparable with, essentially equivalent to, or equivalent to the surface finish of a silicon wafer suitable for semiconductor production.

In another aspect, a device is provided that comprises (i) a composite, solid support member comprised of a synthetic polymeric substrate with an upper surface having a high-quality surface finish and a lower surface, the upper surface comprising a bilayer or multilayer coating comprised of at least a reflective layer deposited on the upper surface and at least a dielectric, transparent layer deposited on the reflective layer, and (ii) a plurality of binding members immobilized to the composite, solid support member (e.g., immobilized to the lower surface).

In another aspect, a kit for detecting a biological analyte of interest in a test sample is provided. The kit comprises an assay comprising a detection zone, the detection zone comprising a composite, solid support member comprised of a synthetic polymeric substrate with an upper surface and a lower surface, the upper surface comprising a bilayer coating or a multilayer coating, said coating being comprised of at least one reflective layer deposited on the upper surface and at least one dielectric, transparent layer deposited on the reflective layer, and a plurality of binding members immobilized to the composite, solid support member, (ii) a container comprising a population of detectable plasmonic particles, and (iii) instructions for use.

In another aspect, a method for the detection of a biological analyte in a fluid sample is provided. The method comprises (i) contacting a device described herein with a fluid sample suspected of comprising the biological analyte of interest and with a detectable particle associated with a binding species for the analyte of interest, where the device comprises an immobilized member with binding for the binding species, and (ii) analyzing the device with an optical instrument for presence or absence of the detectable particle.

In another aspect, a method for the detection of a biological analyte in a fluid sample is provided. The method comprises contacting a device described herein with a fluid sample suspected of comprising the biological analyte of interest and with a detectable particle associated with a binding species for the analyte of interest, where the device comprises an immobilized member with binding for the binding species, and analyzing the device with a dark field optical microscope or dark-field spectrophotometer, which comprises (i) a light beam emitter directed on a sample through a first optical path, (ii) an array of photodetectors arranged on an axis orthogonal to the sample surface (i.e., the sensor surface and sample surface are parallel) and configured to detect light reflected through a second optical path, wherein the photodetectors are not in the path of reflected light (i.e., a second optical path) and the first and second optical paths do not coincide, (iii) one or more optical objectives configured to gather light detected by the photodetectors, and (iv) a processor for the light beam received by the photodetectors, said processor correlating each photodetector to a spatial point on the sample such that measurements are carried out sequentially across various wavelengths and in parallel along X-Y spatial coordinates. In certain embodiments, the light beam emitter is a collimated light beam emitter, the first optical path has at least one lens or an array of lenses which sequentially illuminate the sample at various wavelengths. In embodiments where the light beam emitter is an LED emitter, the first optical path may employ condenser optics and collimation optics. In embodiments using a LASER system, lenses are not needed. Preferably, the light beam is a collimated light beam.

In an aspect, a photodetector described herein comprises an optical objective that achieves an optical resolution of at least about 3-4 microns (i.e., the minimum optical resolution needed to identify and/or classify nanoparticles). In an aspect, the second optical path cannot coincide with the first optical path. In certain embodiments, the axis of the detection module is orthogonal to the sample surface to be detected.

In another aspect, a method for the detection of a biological analyte in a fluid sample is provided. The method comprises contacting a device described herein with a fluid sample suspected of comprising the biological analyte of interest and with a detectable particle associated with a binding species for the analyte of interest, where the device comprises an immobilized member with binding for the binding species, and analyzing the device with a dark field optical microscope or dark-field spectrophotometer, which comprises (i) a white light beam emitter directed on a sample through a first optical path having at least one lens or an array of lenses, thereby illuminating the sample at all wavelengths, (ii) an array of photodetectors arranged to detect light reflected through a second optical path defined as the path of the light beam after reflecting on the sample which can distinguish between different range of wavelengths, and (iii)) one or more optical objectives configured to gather light detected by the photodetectors, and (iv) a processor for the image received by the photodetector, said processor correlating each photodetector to a spatial point on the sample such that measurements are carried out sequentially across various wavelengths and in parallel along X-Y spatial coordinates. In an aspect, the first and second optical paths do not coincide. In certain embodiments, the light beam emitter is a collimated light beam emitter, and the first optical path has at least one lens or an array of lenses which sequentially illuminate the sample at various wavelengths. In embodiments where the light beam emitter is an LED emitter, the first optical path may employ condenser optics and collimation optics. In embodiments using a LASER system, lenses are not needed. Preferably, the light beam is a collimated light beam.

In another aspect, a method for the detection of a biological analyte in a fluid sample is provided. The method comprises contacting a device described herein with a fluid sample suspected of comprising the biological analyte of interest and with a detectable particle associated with a binding species for the analyte of interest, where the device comprises an immobilized member with binding for the binding species, and analyzing the device with a dark field optical microscope or dark-field spectrophotometer, which comprises (i) multiple light emitters or a light emitter configured to emit multiple beams, wherein said light emitter(s) is/are directed on a sample through a first optical path, thereby illuminating the sample at specific wavelengths sequentially with a collimated light beam emitter, (ii) an array of monochromatic photodetectors arranged on to detect light reflected through a second optical path defined as the path of the light beam after reflecting on the sample that can collect the light reflected for each wavelength, and (iv)) one or more optical objectives configured to gather light detected by the photodetectors, and (v) a processor for the light beam received by the photodetectors, said processor correlating each image received to a spatial point on the sample such that measurements are carried out sequentially across various wavelengths and in parallel along X-Y spatial coordinates. In an aspect, the first and second optical paths do not coincide. In certain embodiments, the light beam emitter is a collimated light beam emitter, and the first optical path has at least one lens or an array of lenses which sequentially illuminate the sample at various wavelengths. In embodiments where the light beam emitter is an LED emitter, the first optical path may employ condenser optics and collimation optics. In embodiments using a LASER system, lenses are not needed. Preferably, the light beam is a collimated light beam.

In certain embodiments, the optical instrument may comprise, for example, a complementary metal oxide semiconductor (CMOS) sensor, such as an RGB CMOS sensor.

In embodiments, the optical instrument is a microscope spectrophotometer for dark field measurements, which comprises (i) a light beam emitter directed on a sample through a first optical path having an array of lenses, thereby illuminating the sample at all wavelengths, (ii) a set of filters that can select a subset of light wavelengths, (iii) an array of photodetectors arranged to detect light reflected through a second optical path defined as the path of the light beam after reflecting on the sample that can collect the light reflected, (iv)) one or more optical objectives configured to gather light detected by the photodetectors, and (v) a processor for the light beam received by the photodetectors, said processor correlating each image received to a spatial point on the sample such that measurements are carried out sequentially across various wavelengths and in parallel along X-Y spatial coordinates. In an aspect, the first and second optical paths do not coincide. In certain embodiments, the light beam emitter is a collimated light beam emitter, and the first optical path has at least one lens or an array of lenses which sequentially illuminate the sample at various wavelengths. In embodiments where the light beam emitter is an LED emitter, the first optical path may employ condenser optics and collimation optics. In embodiments using a LASER system, lenses are not needed. Preferably, the light beam is a collimated light beam.

In addition to the exemplary aspects and embodiments described above, further aspects and embodiments will become apparent by reference to the drawings and by study of the following descriptions.

Additional embodiments of the present devices, kits and methods, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1A shows the optical performance of an exemplary device used in an AVAC 50X analyzer, which detects plasmonic nanoparticles by measuring the weak scattering signal with dark-field micro-spectrophotometry. AVAC technology is described in, for example, United States Patent Application Publication No. 2020-0319102, United States Patent Application Publication No. 2020-0319085, and U.S. Pat. No. 10,281,330, each of which is incorporated herein by reference.

FIG. 1B shows that monomers can be identified using AVAC analysis.

FIG. 2 shows the optical performance of another coated cyclic olefin polymer (COP) substrate device

FIGS. 3A-3B show the signal-to-noise ratio (SNR) achieved using devices having a first reflective layer which is either 100 nm (FIG. 3A) or 50 nm thick (FIG. 3B). As used herein, the signal-to-noise ratio (SNR) means the ratio of the scattering signal of the nanoparticle (i.e., the signal of interest) to the scattering signal coming from the surrounding substrate (i.e., the signal not of interest, or “noise”).

FIGS. 4A and 4B show that cyclic olefin polymer (COP) disk embodiments having aluminum first layers and silicon dioxide second layers were not damaged or degraded after 20 hours of incubation in water (FIG. 4A) or carbonate (FIG. 4B), irrespective of the presence of a (3-glycidyloxypropyl)trimethoxysilane (GPTMS)overcoating.

FIGS. 5A-5E show the results of degradation tests performed on various substrates coated wither either aluminum, copper, or gold. Each substrate described in FIGS. 5A-5E is coated with a 50 nm layer of aluminum, as well as a silicon oxide coating of 50 nm.

FIG. 6 describes the optical performance of COP substrates coated with a silicon dioxide layer of varying thickness in the presence or absence of GPTMS. Each substrate described in FIG. 6 is coated with a 50 nm layer of aluminum, whereas the thickness of silicon oxide varies.

FIGS. 7A-7C describe the optical performance of COP substrates coated with a silicon dioxide layer of varying thickness.

FIGS. 8A and 8B describe the roughness of uncoated COP substrates obtained using either high quality molds (steel polished molds) or ultra-high quality molds (steel polished molds with nickel inserts).

FIGS. 9A-9F show surface measurements of various embodiments of COP substrates with a combination of Si and SiO₂.

FIG. 10 shows surface measurements obtained from a reference substrate made of silicon.

FIGS. 11A-11C show the results obtained using aluminum-coated and silicon-coated substrates having varying oxide layer thicknesses.

FIGS. 12A-12C show the results obtained for background scattering, signal and signal to noise ratio, using aluminum-coated and silicon-coated substrates having varying oxide layer thicknesses as compared to reference blanks made of COP or silicon.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. Definitions

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The disjunctive “or” is inclusive, unless otherwise specified. For example, “X or Y” means “X, Y, or both X and Y” unless otherwise specified.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

As used herein to describe a substance or material, the term “dielectric” means that the substance or material is an electrical insulator that can be polarized by an applied electric field (i.e., when the substance/material is placed in an electric field, electric charges do not flow through the substance/material as they do in an electrical conductor because the substance/material has no loosely bound or free electrons). Rather, the electrons shift only slightly from their average equilibrium positions, thereby causing the dielectric polarization. Positive charges are displaced in the direction of the field and negative charges shift in the direction opposite to the field.

As used herein to describe substrates, the term “reflective” means that the substrate can reflect electromagnetic radiation at a reflectivity of at least 10%, or at least 20%, or at least 30%, or at least 40%, or at least 50%, or at least 60%, or at least 70%, or at least 80%, or at least 90% of radiation provided. In certain, non-limiting embodiments, the radiation is visible light radiation or near-IR or IR radiation provided onto the reflective substrate surface, preferably in a specular manner. The radiation (i.e., illumination) may be provided, for example and without limitation, at an angle of less than 90 degrees, relative to the substrate surface. For example, the angle of illumination may be in the range of about 1-89 degrees, or about 10-80 degrees, or about 15-75 degrees, or about 20-70 degrees, or about 25-65 degrees, or about 30-60 degrees. For example and without limitation, provided radiation may be of a wavelength in the range of 300-1000 nm, or in the range of visible light (i.e., about 350 nm to about 850 nm, or about 400 nm to about 825 nm, or about 450 nm to about 800 nm, for example).

As used herein, the term “substrate” (or “solid substrate”) means an object or substance having an ultra-high quality surface roughness and which can be used as a support or base for receiving on one surface thereof materials for a bioassay, such as the coating materials and immunoassay materials described herein. In an aspect, a substrate described herein has an upper surface comparable to the roughness of a silicon wafer used in a semiconductor application when measured using the same technique. In an embodiment, the surface roughness is measured with a regular atomic force microscopy probe and, in embodiments, the measured roughness is less than about 2 nm or less than about 1 nm. Generally, the substrate is solid object and is not magnetic. The substrate can have any shape depending on the desired application, for example the substrate may be provided as a planar substrate, though the substrate can have any useful shape or configuration.

As used herein, the term “transparent” means that a surface is non-reflective. A substrate surface is “non-reflective” if, for example, the surface has a reflectivity of less than 10%, less than 9%, less than 8%, less than 7%, less than 6%, less than 5%, less than 4%, less than 3%, less than 2% or less than 1% of provided electromagnetic radiation, such as provided visible light.

Reflectivity at, for example, wavelengths from 400 nm to 1000 nm, means that the composite substrate has a reflectivity greater than a specified amount at all wavelengths between 400 nm and 1000 nm. The reflectivity of the reflective substrate can be determined at an angle as described above, using, e.g., a reflectometer equipped with a multi-wavelength light source and a spectrometer.

As used herein with respect to a substrate or support, the terms “immobilizing” or “immobilized” include covalent conjugation, non-specific association, ionic interactions and other means of adhering a substance (e.g., a polymer, a copolymer, a binding moiety) to a substrate or support, i.e., a surface of a substrate or support.

As used herein, the term “antibody” means a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, for example, as intact immunoglobulins or as a number of well characterized fragments originally produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. The “Fc” portion of an antibody refers to that portion of an immunoglobulin heavy chain that comprises one or more heavy chain constant region domains, CH₁, CH₂ and CH₃, but does not include the heavy chain variable region.

The term “detectable response” as used herein refers to a change in or an occurrence of a signal that is directly or indirectly detectable either by observation or by instrumentation and the presence of or the magnitude of which is a function of the presence of a target analyte of interest in a test sample. Typically, the response is plasmonic detectable response. Typically, the detectable response is an optical response from a particle, such as a metal particle or a fluorophore (e.g., a plasmonic particle such as a plasmonic nanoparticle), due to its localization in an array or resulting in a change in the wavelength distribution patterns or intensity of reflectance, absorbance or fluorescence or a change in light scatter, fluorescence quantum yield, fluorescence lifetime, fluorescence polarization, a shift in excitation or emission wavelength or a combination of the above parameters. The detectable change in a given spectral property is generally an increase or a decrease and can also be a shift in a spectral measure.

As used herein, the term “metalloid” means a chemical element recognized by a person of ordinary skill in the art as having properties that are in between or that are a mixture of properties of metals and nonmetals metal, including alloys containing at least one such element, and/or a compound containing at least one such element. In embodiments, the metalloid employed is selenium, boron, silicon, germanium, arsenic, antimony, tellurium, and/or polonium, or one or more compounds or alloys thereof. In other embodiments, the metalloid is boron, silicon, germanium, arsenic, antimony and/or polonium, or one or more oxides thereof.

The devices, kits and methods of the present disclosure can comprise, consist essentially of, or consist of, the components or steps disclosed.

All ranges disclosed herein include all subranges contained therein, as well as all discreet values contained therein. Additionally, all ranges disclosed herein are inclusive of their endpoints, unless otherwise specified. For example, “X to Y” means “greater than or equal to X and less than or equal to Y” unless otherwise specified.

When used to describe the amounts of components of a composition, all percentages, parts and ratios are based upon the total weight of the composition, unless otherwise specified.

All measurements made are at about 25° C., unless otherwise specified. Additionally, all measurements made are at about 1 atm of pressure, unless otherwise specified.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Composite Substrate and Devices

Provided herein are solid supports, devices and methods for use in bioanalytical operations. Embodiments of the supports and devices find use in assays for capture of biomolecules or analytes in a test sample, including assays for nucleic acid hybridization, protein interaction, antibody binding, and other analytical assays. The solid supports, devices and methods provide for fast, sensitive, reliable, and/or, optionally, multiplexed detection of biomolecules and other compounds present in biological samples. The devices and methods are intended for use, for example, in research, clinical laboratories, medical clinics, hospital clinics, retirement homes, outpatient clinics, emergency rooms, individual point of care situations (doctor's office, emergency room, out in the field, etc.), and high throughput testing applications.

Devices, methods and kits described herein comprise or utilize a solid substrate or support (e.g., a composite, solid support member) having a base layer and a coating on the base layer, wherein the coating comprises at least one layer.

In certain embodiments, a device described herein may comprise an inlet port and/or an outlet port and/or one or more chambers (e.g., mixing chambers, waste reservoirs and the like). In certain embodiments, one or more channels may be provided to connect ports and/or chambers provided in the device.

A. Substrate

The substrate may be comprised of, consist of, or consist essentially of a polymer or copolymer. For example, the substrate comprises a thermoplastic material. In embodiments, the substrate may comprise one or more materials selected from the group consisting of styrene/methyl methacrylate (SMMA) copolymers, polymethylmethacrylates (PMMAs), olefins, polyesters, polystyrenes, polyethylenes, polyamides, acrylonitrile butadiene styrenes, and polyacetals. In embodiments, the substrate comprises a cyclic olefin copolymer and/or a cyclic olefin polymer (COP).

In some embodiments, the substrate is a disk, such as a COP disk. In other embodiments, the substrate is a slide. In other embodiments the substrate may take other shapes as needed to interface with fluidics or other ways of introducing a sample to it. It may be a part of a larger device and may be enclosed or attached to it

In other embodiments, the substrate may be silicon, or combinations of silicon with thermoplastic materials

The substrate may have a thickness in the range of from about 100 microns to about 1.2 mm, or from about 300 microns to about 700 microns, or from about 700 microns to about 1 mm.

In certain preferred embodiments, the substrate has a thickness of less than about 1.0 mm, less than about 0.9 mm, or less than about 0.8 mm, or less than about 0.7 mm, or less than about 0.6 mm, or less than about 0.5 mm, or less than about 0.4 mm, or less than about 0.3 mm, or less than about 0.2 mm, or less than about 0.1 mm.

The substrate has an upper surface which smooth or essentially smooth, exhibiting very low roughness, i.e., a very high quality surface finish (e.g., essentially equivalent to the roughness of a silicon wafer).

Additionally, the overall shape of the substrate is preferably planar or essentially planar.

Preferably, in an embodiment, the substrate is rigid or essentially rigid.

In embodiments, the substrate has a surface roughness (i.e., on one or both of an upper surface and/or a lower surface of the substrate) which is equivalent or essentially equivalent to a silicon wafer suitable for use in semiconductor production or as a semiconductor.

In certain embodiments, the surface finish (roughness) of the substrate is equivalent or essentially equivalent to the surface finish (roughness) of a silicon wafer before a coating has been applied.

In other embodiments, the surface finish (roughness) of the substrate is equivalent or essentially equivalent to the surface finish of a silicon wafer after a coating has been applied.

In yet other embodiments, the surface finish (roughness) of the substrate is equivalent or essentially equivalent to the surface finish of a silicon wafer before and after a coating has been applied. That is, in such embodiments, the application of a coating on the substrate does not impact the overall surface finish of the substrate/coating composite relative to the uncoated substrate. In these embodiments, the technique used to measure surface roughness of the substrate is the same as the technique used to measure the surface roughness of the silicon wafer.

It has been found that maintaining a smooth or essentially smooth substrate surface (or coated substrate surface) minimizes the amount (i.e., intensity) of reflection resulting from an imperfect (rough) top surface, as well as the number of reflections resulting from an imperfect bottom surface, as transmitted light passes through the substrate and hits a surface beneath the substrate. The surface beneath the substrate may have a surface finish or roughness which is difficult to control, thus emphasizing the need to maintain a smooth substrate surface to compensate for other reflections

An ideal substrate surface having essentially perfect surface finish (i.e., roughness) would maximize the contrast between background and detected particles when analyzed via, e.g., dark field microscopy, or another analytical method. A rough substrate, on the other hand, would produce a diffuse or noisy background, thereby reducing the contrast between background and particles. This effect is especially relevant in dark field imaging such as dark field microscopy, as the scattered light of interest (for example, from nanoparticles) accounts for a very small fraction of illumination. Thus, any additional source of light scattering will very likely be in the same range as the signal.

An ordinarily skilled person will recognize that surface finish (i.e., roughness) can be measured using various methods. It is understood that the parameter Ra is the universal, most widely used parameter for roughness internationally. Ra is the arithmetic mean of the departure of a surface's roughness profile from a mean line (i.e., a reference line representing the overall surface). A hypothetical substrate's surface roughness of 10 nm therefore means that such a substrate has a mean (average) departure of 10 nm from its overall surface, as measured across the entire surface of the substrate. In an aspect, a substrate described herein has an upper surface having a surface roughness of about 0-100 nm, or about 0-75 nm, or about 0-50 nm, or about 0-25 nm, or about 0-10 nm. In certain embodiments, the upper surface has a roughness comparable or equivalent or essentially equivalent to the roughness of a silicon wafer which is suitable for semiconductor production.

Additionally, the substrate is planar or essentially planar. As used herein, “flatness” is a measure or indication of a surface's warpage or deviation from being planar (with a zero value indicating perfect flatness). For example, the surface(s) of the substrate may have a flatness in the range of from about 0 μm to about 100 μm, or from about 0 μm to about 90 μm, or from about 0 μm to about 80 μm, or from about 0 μm to about 70 μm. For example, the flatness of a composite described herein may be about 0 μm, or about 5 μm, or about 10 μm, or about 15 μm, or about 20 μm, or about 25 μm, or about 30 μm, or about 35 μm, or about 40 μm, or about 45 μm, or about 50 μm, or about 55 μm, or about 60 μm, or about 65 μm, or about 70 μm, or about 75 μm, or about 80 μm, or about 85 μm, or about 90 μm, or about 95 μm, or about 100 μm. Preferably, the flatness is minimized, i.e., less than 100 μm and more preferably approximately 0 μm.

In the case of an aluminum-coated substrate, such as an aluminum-coated COP substrate, the flatness may be from about 0 μm to about 90 μm, or about 05 μm to about 85 μm, or about 0 μm to about 80 μm, or from about 0 μm to about 75 μm.

For example, one or both of the upper and/or lower substrate surfaces may be produced using polished steel mold or through the addition of nickel inserts or alternative techniques that cover different or varying levels of roughness and are suitable for different surface quality (e.g., absence of polish-related scratches).

In certain embodiments, the substrate incorporates a ferromagnetic metal, which allows for remote surface magnetization in the substrate. In certain embodiments, the ferromagnetic metal is nickel or cobalt, or an alloy comprising nickel and/or cobalt. For example, nickel vanadium may be used as a ferromagnetic metal or ferromagnetic additive.

In an aspect, a ferromagnetic metal or alloy may be provided in any layer on the coated substrate. In certain embodiments, the substrate is directly coated with a ferromagnetic metal or alloy. The thickness of a ferromagnetic metal or alloy layer may be, e.g., from 100 nm to 200 nm thick. Subsequent layers (e.g., a reflective or substantially reflective layer, an overcoat layer, or other layer described herein) may be provided on the ferromagnetic metal or alloy layer.

For example and without limitation, a device described herein may comprise a ferromagnetic layer which is about 100-200 nm thick, a reflective layer having a thickness described herein, and an optional silicon dioxide layer having a thickness described herein. The reflective layer may be achieved using aluminum or another metal, or may be achieved using stacked dielectric materials with alternating high and low refractive indexes.

B. Coating

The coating comprises at least one layer (i.e., a first layer) which is reflective or substantially reflective of an electromagnetic radiation. In embodiments, the first layer of the coating reflects or substantially reflects visible light

In embodiments, the coating comprises at least one layer (i.e., a first layer) that comprises one or more metals or metalloids selected from aluminum, silver, gold, chromium, nickel, cobalt and silicon, and alloys and compounds containing one or more of aluminum, silver, gold, chromium, nickel, cobalt or silicon. In other embodiments, the first layer may be comprised of stacked dielectric materials with alternating high and low refractive indexes. In other embodiments, the first layer comprises a metal and/or metalloid, as well as one or more dielectric materials.

In embodiments, the first layer of the coating consists essentially of a metal or metalloid selected from aluminum, silver, gold, chromium, nickel, cobalt and silicon. In other embodiments, the first layer of the coating consists of a metal or metalloid selected from aluminum, silver, gold, chromium, and silicon.

In embodiments, the coating comprises two or more layers. In certain preferred embodiments, the coating is a bilayer, i.e., a coating comprising or consisting of a reflective first layer as described above and a transparent second layer.

In embodiments, the second layer of the bilayer coating is dielectric and transparent.

Preferably, the second layer of the bilayer coating can be functionalized (e.g., functionalized to a detectable particle). More preferably, the assay is a “sandwich” type assay wherein the second layer is functionalized to a capture portion, whereas the detectable particle is part of the detection portion.

The second layer of the bilayer coating may be comprised of a material selected from metalloids, alloys and compounds containing one or more metalloids, and polymers. In embodiments, a polymer used as the second layer is a synthetic polymer.

Where the second layer is a metalloid, alloy of a metalloid or a compound of a metalloid, the metalloid is selected from the group consisting of selenium, boron, silicon, germanium, arsenic, antimony, tellurium, and polonium.

Preferably, the metalloid is one or more of boron, silicon, germanium, arsenic, antimony and/or polonium. Where the second layer is a compound containing one or more metalloids, the compound is preferably an oxide of a metalloid. For example, in certain preferred embodiments, the second layer is an oxide of silicon, preferably silicon dioxide (which may be applied via any suitable means of application, including but not limited to chemical vapor deposition (CVD) or remote combustion chemical vapor deposition (r-CCVD)).

In embodiments containing a bilayer coating, the first layer (i.e., the reflective layer) may have a thickness in the range of from about 10 nm to about 1,000 nm, or in the range of from about 10 nm to about 500 nm, or in the range of from about 10 nm to about 250 nm, or in the range of from about 10 nm to about 200 nm, or in the range of from about 50 nm to about 150 nm, or in the range of from about 75 nm to about 125 nm.

For example, a first layer (i.e., a reflective layer) may have a thickness in the range of from about 1 nm to about 250 nm, or in the range of from about 25 nm to about 250 nm.

In embodiments containing a bilayer coating, the second layer (i.e., a transparent layer or a dielectric transparent layer) may have a thickness in the range of from about 10 nm to about 500 nm, or in the range of from about 20 nm to about 200 nm, or in the range of from about 20 nm to about 100 nm, or in the range of from about 50 nm to about 100 nm, or in the range of from about 75 nm to about 100 nm, or in the range of from about 70 nm to about 90 nm.

For example, a second layer (i.e., a transparent layer or a dielectric transparent layer) may have a thickness in the range of from about 1 nm to about 250 nm, or in the range of from about 25 nm to about 250 nm.

In certain preferred embodiments having a bilayer coating, the first layer and the second layer have a thickness within about 30% of each other, 25% of each other, 20% of each other, 15% of each other, or 10% of each other.

In certain preferred embodiments, the first layer and the second layer are the same thickness or approximately the same thickness (e.g., within about 5% of each other).

In other preferred embodiments, the first layer has a thickness greater than that of the second layer.

The device may optionally contain an additional layer applied to the second layer after the second layer is applied to the first layer. Said additional layer (or “overcoating” or “overcoat layer”) is preferably applied with an epoxy silane, such as (3-glycidyloxypropyl)trimethoxysilane (“GPTMS”) or a chlorosilane such as 3-chloropropyl triethoxysilane, or 3-aminopropyltrimethoxysilane (APTMS), or 3-aminopropyltriethoxysilane (APTES).

In embodiments of the composite, solid support, the reflective layer (i.e., a first layer) comprises or consists essentially of aluminum and the transparent dielectric layer (i.e., a second layer) comprises or consists essentially of silicon dioxide.

In other embodiments of the composite, solid support, the reflective layer (i.e., a first layer) consists of aluminum and the transparent dielectric layer (i.e., a second layer) consists of silicon dioxide.

In embodiments of the composite, solid support, the coating comprises silicon dioxide and APTMS. In embodiments of the composite, solid support, the coating consists essentially of silicon dioxide and APTMS.

In embodiments of a device described herein, the substrate is silicon, monocrystalline silicon or a silicon wafer.

In some embodiments, the coating is a 20 nm silicon dioxide layer on a silicon wafer. In other embodiments, the coating is silicon dioxide with a thickness of greater than 20 nm. In still other embodiments, the

In an aspect, manufacturing with high quality molds (i.e., molds including nickel inserts) and deposition of the one or more layers on the polymeric substrate achieves a device which replaces silicon-based, ultra-flat chips. The device described herein thus represents an improvement over more expensive ultra-flat silicon chips, which require more complicated manufacturing methods.

In embodiments, a device described herein may be configured to comprise an inlet port and/or an outlet port.

In one aspect, provided is a device comprising a synthetic polymeric substrate having an upper surface, the upper surface having a coating which comprises a first layer comprised of a material that reflects electromagnetic radiation and a second layer comprised of a material that is dielectric and transparent.

In another aspect, a device comprises a substrate having an upper surface on which a coating is provided. In embodiments, the coating has at least two layers, the first of which is provided directly on the upper surface of the substrate, and the second layer being provided on the provided first layer. The first layer is reflective and the second layer is transparent such that light (e.g., visible light) may pass through the second layer and be reflected back through the second layer by the first layer.

In another aspect, a device comprises a substrate having an upper surface on which a coating is provided, the coating being of the first and second layers as described above, as well as an overcoating (overcoat layer) provided on the second layer after the second layer is provided on the first layer (which was itself provided on the substrate). In certain preferred embodiments, the overcoating is APTMS or GPTMS.

As described herein, a device may further comprise a plurality of binding members immobilized on the composite support member (e.g., on the second layer or on the overcoating, if present).

C. Binding Members

Additionally, a device described herein may comprise one or more binding members (e.g., a plurality of binding members) which are immobilized onto the composite, solid support member. The composite support member comprises a substrate and one or more of the layers described herein. In a preferred embodiment, the composite support member comprises a substrate having a high quality surface finish as described herein, a first reflective layer and a second transparent layer, wherein the second transparent layer is optionally dielectric. In another preferred embodiment, the composite support member comprises a substrate, a first reflective layer and a second transparent layer, wherein the second transparent layer is optionally dielectric, and optionally an overcoating layer (e.g., APTMS or GPTMS).

The binding members may be of the same or different identities. For example, in a device described herein, the plurality of binding members provided on the composite, solid support member may consist of a binding member for a single analyte or single class of analyte. In another embodiment, the plurality of binding members provided on the composite, solid support member may comprise members which bind a first analyte and members which bind a second analyte, and optionally members which bind a third, fourth, fifth analyte or class of analyte or more than five different analytes or classes of analytes.

In a preferred embodiment, the plurality of binding members comprise a first binding member for a first analyte and a second binding member for a second analyte.

The plurality of binding members may comprise a protein, an antibody, or a peptide.

In certain, non-limiting embodiments, the protein may be streptavidin.

In certain, non-limiting embodiments, the antibody is an anti-IL6 antibody.

The binding members provided on the composite, solid support member may optionally comprise a binding tag. In embodiments, the binding tag is a haloalkane dehalogenase or an avidin.

The plurality of binding members comprises a ligand with specific binding for a binding tag that is part of a fusion protein comprising an antibody or antibody fragment that binds an analyte of interest.

In certain, non-limiting embodiments, the binding tag may be a haloalkane dehalogenase or an avidin in embodiments.

In certain, non-limiting embodiments, the ligand may be a synthetic organic compound, such as a chloroalkane linker, or a cross linker.

For instance, the binding tag may be provided using HaloTag™

In certain, non-limiting embodiments, the binding tag may be an enzymatically modified protein or peptide for installing a single protein or peptide. For instance, enzymatic modification can be achieved using an enzyme such as, but not limited to, biotin ligase to achieve biotinylation of a desired protein or peptide. For instance, the binding tag may be provided using AviTag™.

For example, in embodiments, a device comprising (a) a composite, solid support member comprised of a synthetic polymeric substrate with an upper surface having a high quality surface finish, and a lower surface, the upper surface comprising a bilayer coating comprised of (i) a reflective layer deposited on the upper surface and (ii) a dielectric, transparent layer deposited on the reflective layer, and (b) a plurality of binding members immobilized to the composite, solid support member is provided.

In other embodiments, a device comprises (a) a composite, solid support member comprising a synthetic polymeric substrate with an upper surface and a lower surface, the upper surface comprising a coating comprised of (i) a reflective layer deposited on the upper surface and (ii) a dielectric, transparent layer deposited on the reflective layer, and (iii) an overcoating (overcoat layer) which may optionally comprise or consist essentially of GPTMS or APTMS, and (b) a plurality of binding members immobilized to the composite, solid support member via the overcoating.

In other embodiments, a device comprises (a) a composite, solid support member comprising a synthetic polymeric substrate with an upper surface and a lower surface, the upper surface comprising a coating comprised of (i) a reflective layer deposited on the upper surface that contains ferromagnetic materials like Ni, or Co and (ii) a dielectric, transparent layer deposited on the reflective layer, and (iii) an overcoating (overcoat layer) which may optionally comprise or consist essentially of GPTMS or APTMS, and (b) a plurality of binding members immobilized to the composite, solid support member via the overcoating. The ferromagnetic material can be used to induce a surface mediated magnetic field on the surface (i.e., remote surface magnetization) to attract magnetic particles and the like, accelerating binding to the surface.

### III. Kits

In another aspect, a kit for detecting a biological analyte of interest in a test sample is provided.

In embodiments, the kit includes an assay having a detection zone which comprises a composite, solid support member as described herein.

The composite, solid support member of the detection zone may comprise a synthetic polymeric substrate, for example.

Additionally, the composite, solid support member may have an upper surface and a lower surface. In embodiments, the upper surface of the composite, solid support member comprises a bilayer coating comprised of (i) a first, reflective layer deposited on the upper surface and (ii) a second layer deposited on the reflective layer.

In embodiments of the kit described herein, the composite, solid support has a plurality of binding members immobilized thereto. The plurality of immobilized binding members are capable of binding the analyte of interest or a ligand with specific binding for a binding tag that is part of a fusion protein comprising an antibody or antibody fragment that binds an analyte of interest. For example, the plurality of immobilized binding members are one or more of an antibody, an antibody fragment, or a synthetic organic compound.

In certain embodiments, the ligand having a specific binding for a binding tag is biotin.

In embodiments, the plurality of immobilized binding members may be a synthetic organic compound comprising a chloroalkane linker of the appropriate size, and the binding tag may be a haloalkane dehalogenase.

A kit described herein further includes a container comprising a population of detectable particles.

In particular, the kit comprises at least one further binding member, which is capable of associating with the detectable particles and having specific binding for an analyte of interest. In embodiments, the at least one further binding member is an antibody or antibody fragment.

In embodiments, the binding member is an antibody conjugated to the detectable particle via the sulfhydryl (—SH) group.

In embodiments, the antibody or antibody fragment has specific binding for a cardiac biomarker, an inflammation marker (e.g., interleukins ILx, such as IL-6), a neural cell marker (e.g., Tau and isoforms thereof, or other targets for Alzheimer's and/or Parkinson's disease), a marker associated with one or more infectious diseases (e.g., LAM, p24, chemokine panels, IFN panels, etc.).

For example, the cardiac biomarker is a troponin, such as troponin C (TNNC1 or TNNC2), troponin I (cTnI), or troponin T (cTnT), or high-sensitivity (hs) cTnI. Alternatively, the cardiac biomarker may be B-type natriuretic peptide (BNP) or pro-BNP, or a diagnosis panel.

The detectable particles comprise or consist essentially of a metal, preferably a transition metal or a noble metal.

In embodiments, the detectable particles comprise or consist essentially of one or more metals selected from gold, silver, platinum, palladium, iridium, osmium, rhodium, ruthenium and alloys thereof. In certain embodiments, the detectable particles are nanoparticles having at least a plasmonic material embedded therein (e.g., gold, aluminum, silver or a metamaterial).

In embodiments, the detectable particles consist of a metal selected from gold, silver, platinum, palladium, iridium, osmium, rhodium, and ruthenium.

In embodiments, the detectable particles comprise or consist essentially of gold. In embodiments, the detectable particles consist of gold.

The detectable particles have an average diameter ranging from about 1 nm to about 1500 nm, or from about 25 nm to about 500 nm, or from about 50 nm to about 250 nm or from 100 to 200 nm.

In embodiments, the detectable particles resonate at a wavelength ranging from about 250 nm to about 1000 nm, or about 300 nm to about 950 nm, or about 350 nm to about 900 nm, or about 400 nm to about 850 nm, or about 450 nm to about 800 nm.

In embodiments, the detectable particles have a shell-core structure, wherein the core is magnetic and the shell is a transition metal. In embodiments, the core is iron, an oxide of iron, or an iron alloy. In preferred embodiments, the core is iron or iron (II, III) oxide (i.e., Fe₃O₄). The shell is preferably gold.

In embodiments, the diameter of the magnetic core (i.e., the average magnetic core diameter of a plurality of detectable particles) may be in the range of from about 1 nm to about 300 nm, or from about 25 nm to about 250 nm, or from about 50 nm to about 200 nm, or from about 75 nm to about 150 nm and the thickness of the shell may be in the range of from about 0.5 nm to about 50 nm, or from about 1 nm to about 40 nm, or from about 5 nm to about 30 nm, or from about 10 nm to about 25 nm.

In other embodiments, the diameter of the magnetic core may be in the range of from 0.5 nm to about 60 nm, or from about 1 nm to about 40 nm, or from about 3 nm to about 30 nm, or from about 5 nm to about 25 nm. The shell may have a thickness in the range of from about 1 nm to about 100 nm, or from about 5 nm to about 80 nm, or from about 5 nm to about 60 nm, or from about 10 nm to about 45 nm.

Optionally, an intermediate layer may be provided between the core and shell of the detectable particles (i.e., the intermediate layer may be provided as a first shell between the core and the outer shell). The intermediate layer may be comprised of silica. The diameter of the magnetic core (e.g., the average magnetic core diameter of a plurality of detectable particles) may be in the range of from about 1 nm to about 300 nm, or from about 25 nm to about 250 nm, or from about 50 nm to about 200 nm, or from about 75 nm to about 150 nm. The thickness of the intermediate layer may be in the range of from about 0.5 nm to about 50 nm, or from about 1 nm to about 40 nm, or from about 5 nm to about 30 nm, or from about 10 nm to about 25 nm. The thickness of the shell may be in the range of from about 0.5 nm to about 50 nm, or from about 1 nm to about 40 nm, or from about 5 nm to about 30 nm, or from about 10 nm to about 25 nm. The detectable particle may have a diameter (i.e., an average diameter) in the range of from about 25 nm to about 500 nm, or from about 50 nm to about 450 nm, or from about 75 nm to about 350 nm, or from about 100 nm to about 300 nm.

In other embodiments, the detectable particles do not have a core-shell structure. That is, in such embodiments, the detectable particles consist essentially of a transition metal or alloy thereof. For example, the detectable particles may consist essentially of gold.

In an embodiment, the kit may include instructions for use.

### IV. Analyte Detection Method

Also described herein are embodiments of a method for the detection of a biological analyte in a fluid sample. The method comprises contacting a device described herein with (i) a fluid sample suspected of comprising a biological analyte of interest and (ii) a detectable particle associated with a binding species for the analyte of interest.

The device to be contacted comprises an immobilized member with binding for the binding species. After being contacted by the fluid sample and the detectable particle associated with the binding species, the device is analyzed with an optical instrument for presence of absence of the detectable particle.

In embodiments, the optical instrument is a dark field optical microscope or dark-field spectrophotometer having a plurality of photodetectors. In an aspect, any spectrophotometer may be used. In certain embodiments, the photodetectors are monochromatic photodetectors. In other embodiments, the photodetectors are RGB photodetectors.

In embodiments, the microscope or spectrophotometer is capable of carrying out simultaneous analyses at different points on a single sample (i.e., using a single prepared sample on a substrate as described herein), wherein the analyses can be performed with a high spatial resolution and without requiring a mechanical system for physical scanning of the sample to be analyzed. This may be achieved, for example, by utilizing a dark field optical microscope or dark-field spectrophotometer which has a means of processing light received by two or more (i.e., a plurality) of photodetectors) and one or more optical objectives configured to gather light detected by the photodetectors, wherein the processing means have a correlation in which each photodetector and optical objective corresponds to a different spatial point on the same.

In an aspect, an optical objective described herein has a resolution of about 4 nm or less.

The optical microscope or dark-field spectrophotometer can be made for bright field and dark field applications, both for measurements of reflection or transmission, provided that optical components suitable for each of the techniques are used. In preferred embodiments, the optical instrument is a spectrophotometer for dark field measurements.

The dark field optical microscope or dark-field spectrophotometer may have a light source with a broad spectral band (such as, but not limited to, a white light-emitting LED bulb) with a length selector (such as, but not limited to, one or more monochromators, optical filters, prisms, etc.).

Alternatively, the spectrophotometer may have multiple light sources, each having at a different wavelength. For example and without limitation, the multiple light sources may be multiple LEDs or multiple lasers, or combinations of one or more LEDs and one or more lasers. In such an embodiment, a wavelength selector would not be needed, as they would only need to have means for selecting the LED that illuminates the sample, such that wavelength scanning can be performed by changing from one LED to another.

In embodiments, the spectrophotometer's light beam source comprises a monochromator to selectively control the wavelength sent to the sample such that a light beam of a certain wavelength is emitted. Therefore, a simultaneous analysis at different points on the same sample at the same wavelength may be carried out. Thereafter, another wavelength may be selected with the monochromator such that the sample is sequentially illuminated with several wavelengths.

In embodiments, the optical instrument is a spectrophotometer for dark field measurements, which comprises (i) a light beam emitter directed on a sample through a first optical path having an array of lenses, thereby sequentially illuminating the sample at various wavelengths, (ii) an array of photodetectors arranged to detect light reflected through a second optical path defined as the path of the light beam after reflecting on the sample, (iii) one or more optical objectives configured to gather light detected by the photodetectors, and (iv) a processor for the light beam received by the photodetectors, said processor correlating each photodetector to a spatial point on the sample such that measurements are carried out sequentially across various wavelengths and in parallel along X-Y spatial coordinates.

In embodiments, the optical instrument is a spectrophotometer for dark field measurements, which comprises (i) a white light beam emitter directed on a sample through a first optical path having an array of lenses, thereby illuminating the sample at all wavelengths, (ii) an array of photodetectors arranged to detect light reflected through a second optical path defined as the path of the light beam after reflecting on the sample which can distinguish between different range of wavelengths, (iii) one or more optical objectives configured to gather light detected by the photodetectors, and (iv) a processor for the image received by the photodetector, said processor correlating each photodetector to a spatial point on the sample such that measurements are carried out sequentially across various wavelengths and in parallel along X-Y spatial coordinates.

In an embodiment, the array of photodetectors can distinguish between different wavelengths or ranges of wavelengths at, for example, the red, green, and blue parts of the visible light spectrum.

In certain embodiments, the optical instrument may comprise, for example, a complementary metal oxide semiconductor (CMOS) sensor, such as an RGB CMOS sensor.

In embodiments, the optical instrument is a spectrophotometer for dark field measurements, which comprises (i) a light beam emitter directed on a sample through a first optical path having an array of lenses, thereby illuminating the sample at all wavelengths, (ii) a set of filters that can select a subset of light frequencies, (iii) an array of photodetectors arranged to detect light reflected through a second optical path defined as the path of the light beam after reflecting on the sample that can collect the light reflected, (iv) one or more optical objectives configured to gather light detected by the photodetectors, and (v) a processor for the light beam received by the photodetectors, said processor correlating each image received to a spatial point on the sample such that measurements are carried out sequentially across various wavelengths and in parallel along X-Y spatial coordinates.

In an embodiment, the filters can select a subset of light frequencies in, for example, the red, green, or blue (RGB) portions of the visible light spectrum.

In another aspect, a method for the detection of a biological analyte in a fluid sample is provided. The method comprises contacting a device described herein with a fluid sample suspected of comprising the biological analyte of interest and with a detectable particle associated with a binding species for the analyte of interest, where the device comprises an immobilized member with binding for the binding species, and analyzing the device with a spectrophotometer, which comprises (i) multiple light emitter directed on a sample through a first optical path, thereby illuminating the sample at specific wavelengths sequentially (ii) an array of monochromatic photodetectors arranged on a second optical path defined as the path of the light beam after reflecting on the sample that can collect the light reflected for each wavelength, (iii) one or more optical objectives configured to gather light detected by the photodetectors, and (iv) a processor for the light beam received by the photodetectors, said processor correlating each image received to a spatial point on the sample such that measurements are carried out sequentially across various wavelengths and in parallel along X-Y spatial coordinates.

The spectrophotometer may be configured to take measurements of crossed polarization (provided that appropriate polarizers are coupled along the beam that hits the sample and along the path of the beam that points toward the array of photodetectors). For example, in certain non-limiting embodiments, the array of photodetectors is a CCD camera in which a series of pixels thereof comprises a photodetector. Said series can be one pixel or an array of pixels.

In some embodiments, the source of the light beam comprises a monochromator, such that a light beam of a certain wavelength is emitted. In this way, a parallel analysis at different points on the same sample at the same wavelength is carried out.

Among the different beam sources with a broad spectral band that can be used to carry out the spectrophotometric analysis, the beam source can be a visible, ultraviolet and/or infrared light source.

A spectrophotometer used in a method described herein preferably operates with high sensitivity.

In certain embodiments, a detection method described herein may be configured to detect particles in the femtogram range, or smaller.

In embodiments, a spectrophotometer described in 2020-0319102, US 2020-0319085, and/or U.S. Pat. No. 10,281,330, (which are incorporated herein by reference) may be used. In embodiments, for example, the spectrophotometer operates using AVAC technology as in an AVAC Analyzer (Mecwins).

In embodiments, the spectrophotometer further comprises a dark field microscope objective and a dark field beam splitter.

In an aspect, the method described herein provides improved detection of individual particles in a bioassay. The described method, which uses devices disclosed herein, achieve substantially improved signal-to-noise ratio (SNR) for individual particles (e.g., plasmonic particles) in a bioassay.

In an aspect, the disclosed method detects individual particles with an SNR of at least 60, or of at least 70, or of at least 80, or of at least 90, or of at least 100.

As demonstrated by the below examples, the SNRs obtained using COP-based devices described herein, in combination with dark field microscopy analysis, allow for the detection of individual particles of interest without the need for silicon-based ultra-flat chips/wafers.

### EXAMPLES

Further aspects of the present subject matter will be apparent to persons of ordinary skill in the art based on the following non-limiting Examples.

### Example 1

An exemplary biological assay was performed using a device described herein having a COP substrate and a silicon dioxide (20 nm) layer deposited thereon via physical vapor deposition (PVD). The limit of quantitation of the device used was estimated to be 95 fg/mL.

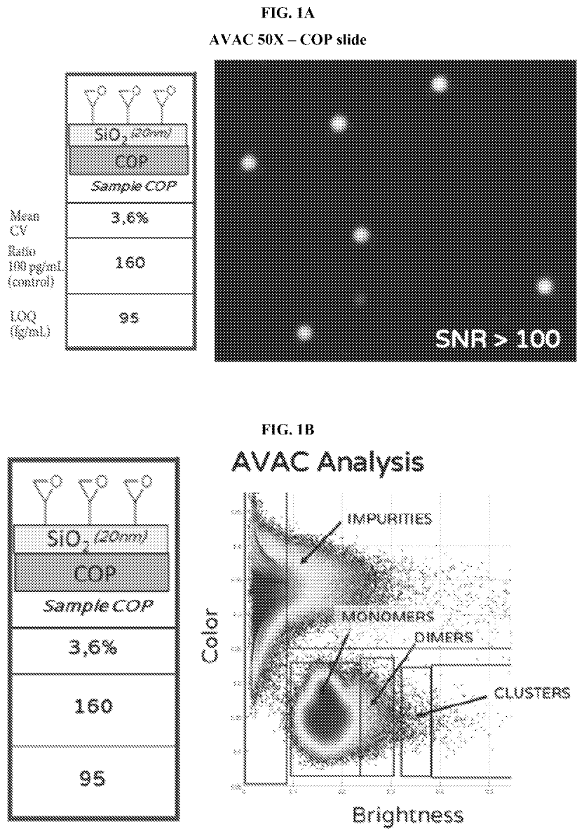

FIG. 1A shows the optical performance of the exemplary device analyzed in an AVAC Analyzer (Mecwins). A signal-to-noise ratio (SNR) of greater than 100 was exhibited.

FIG. 1B shows that monomers can be identified using AVAC analysis in the same device as in FIG. 1A.

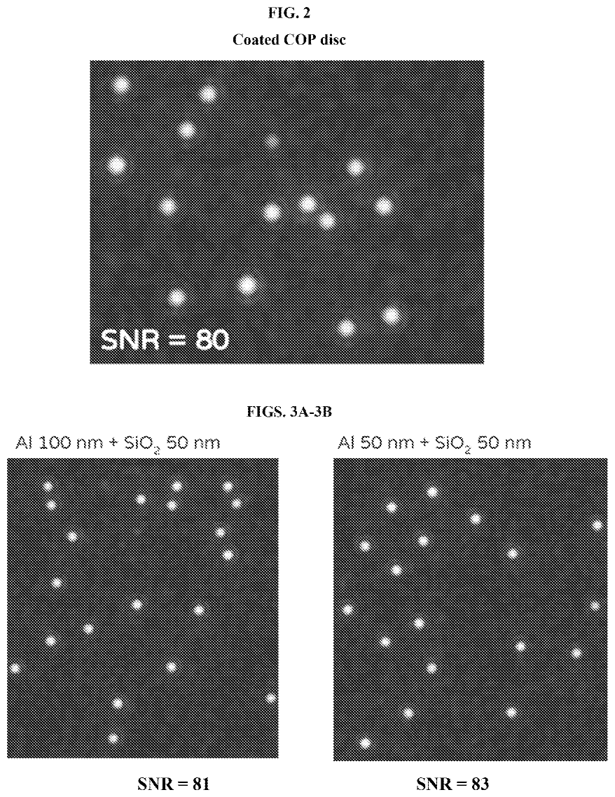

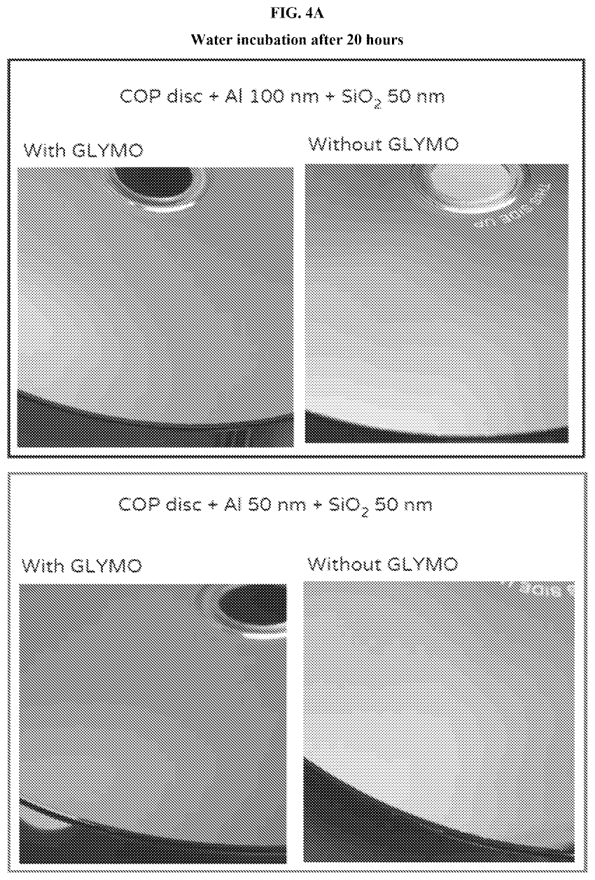

FIG. 2 shows the optical performance of the COP substrate device of FIG. 1B which exhibited an SNR of 80, allowing for the detection of individual particles of interest.

### Example 2

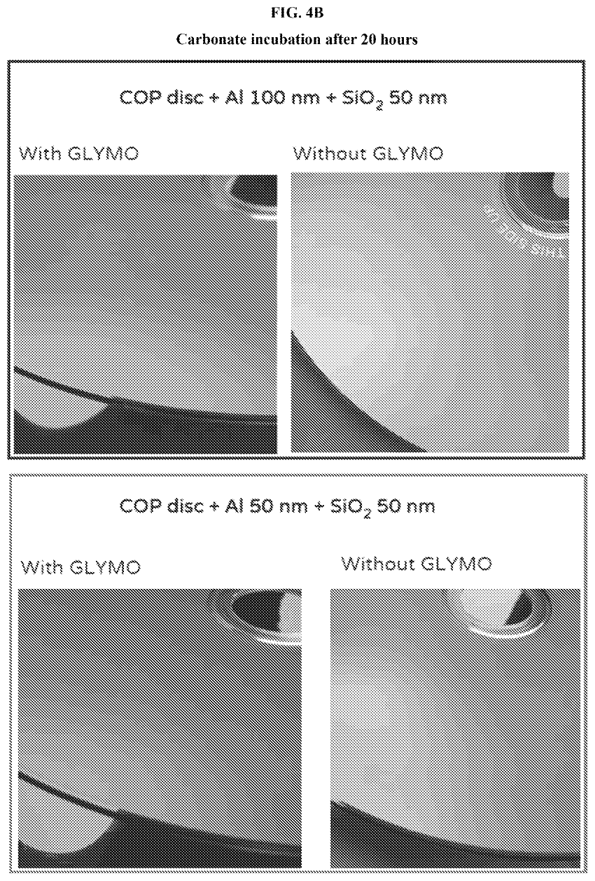

In FIGS. 3A-3B it is shown that reducing the thickness of the first reflective layer from 100 nm (FIG. 3A) to 50 nm (FIG. 3B), while keeping the second transparent layer constant, does not affect optical performance. The two composite, solid supports of the present Example provided similar gold nanoparticle (GNP) detection signals.

The signal-to-noise ratio (SNR) remained essentially unchanged, with the 100 nm aluminum embodiment (FIG. 3A) having an SNR of about 83 and the 50 nm aluminum embodiment (FIG. 3B) having an SNR of about 81.

### Example 3

FIG. 4A shows that COP disk embodiments having aluminum first layers and 50 nm silicon dioxide second layers were not damaged or degraded after 20 hours of incubation in water. FIG. 4B shows that COP disk embodiments having aluminum first layers and 50 nm silicon dioxide second layers were not damaged or degraded after 20 hours of incubation in pH=9 carbonate buffer.

### Example 4

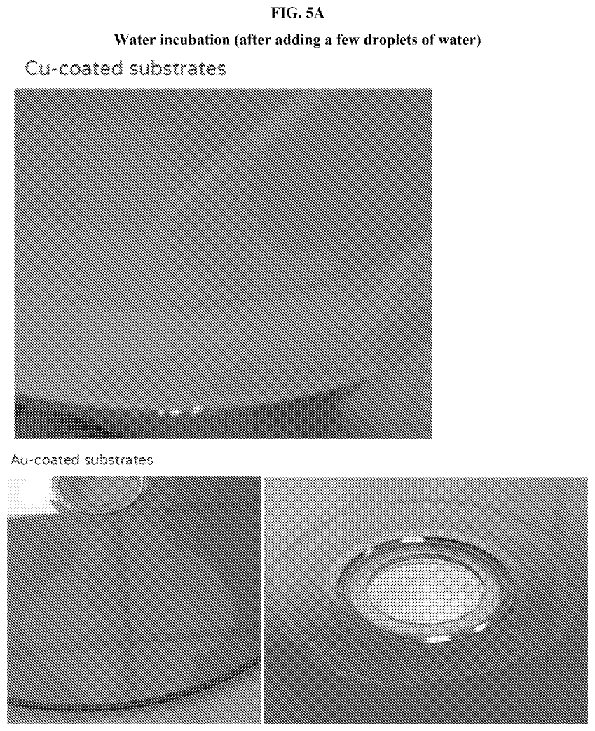

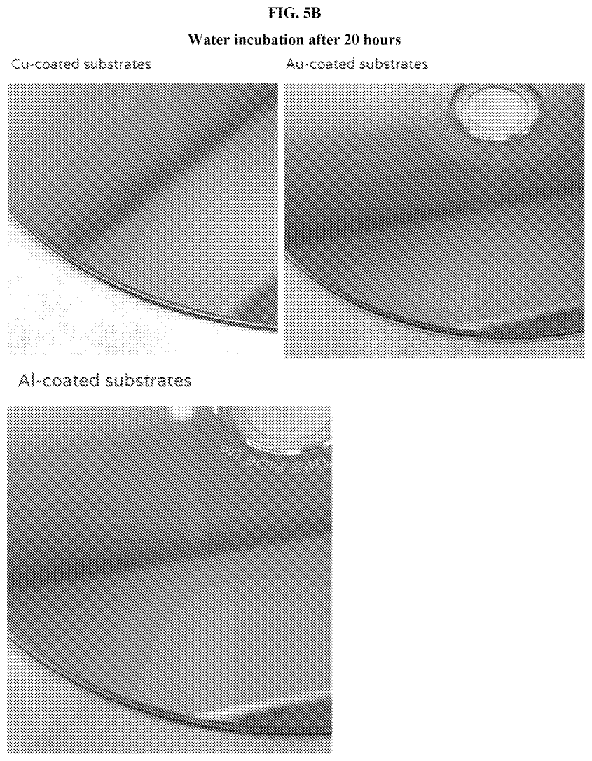

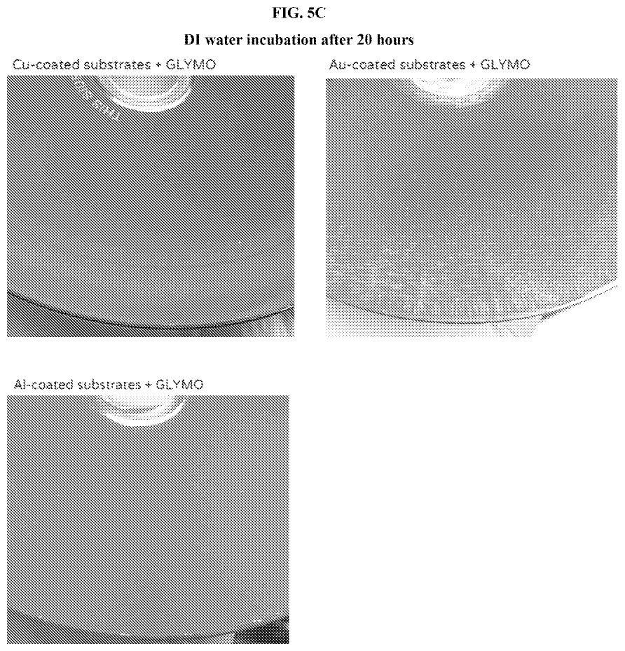

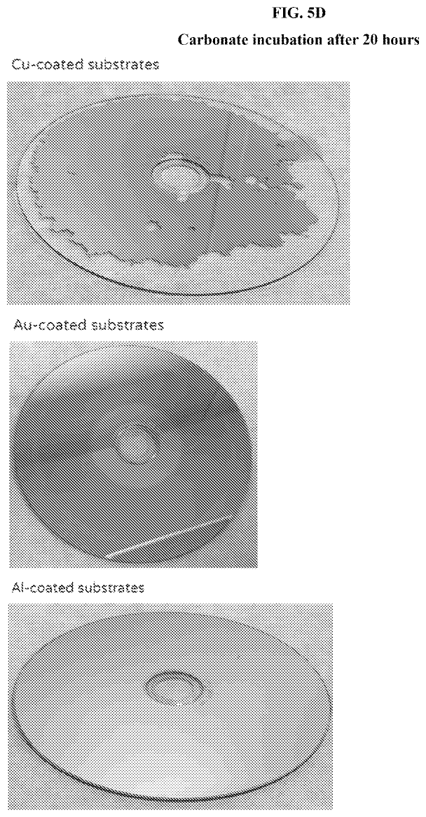

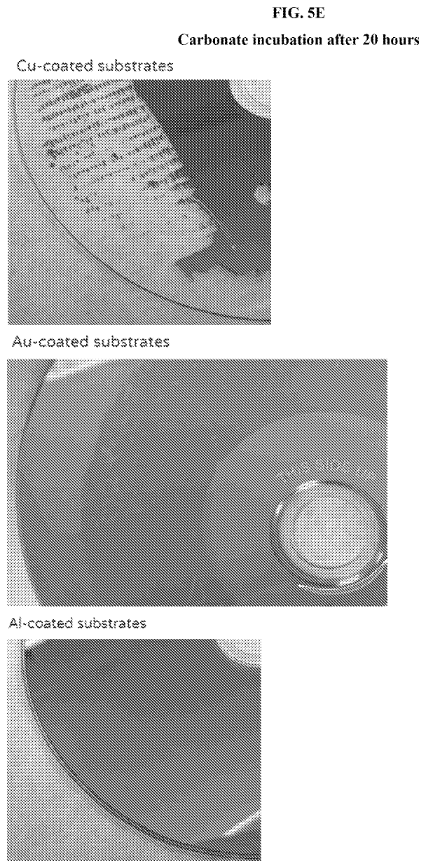

Additionally, aluminum, copper and gold were compared for use in coating COP substrates. The qualitative results of incubating aluminum-, copper-, and gold-coated COP substrates are shown in FIGS. 5A through 5E.

FIG. 5A shows that, after adding only a few droplets of DI water, halos and stains were observed in copper- and gold-coated substrates almost instantaneously upon contact with the water droplets.

By contrast, as shown in FIG. 5B, no damage was observed when aluminum-coated substrates were incubated in water for 20 hours. The copper-coated substrate exhibited haloing at the edges, while the gold-coated substrate was significantly damaged throughout.

FIG. 5C similarly shows that COP disk embodiments having aluminum coating (plus GPTMS) were not damaged after incubation in DI water after 20 hours, whereas embodiments coated with copper or gold were visibly damaged.

In addition to DI water incubation tests, the metal-coated substrates were incubated in carbonate. Again, as shown in FIG. 5D, copper and gold were significantly damaged, whereas the aluminum coating was not visibly damaged after 20 hours of incubation. FIG. 5E shows the results of a peeling test following the carbonate incubation to verify adhesion of the metal coatings. Aluminum showed excellent adhesion, whereas both copper and gold were susceptible to peeling.

### Example 5

Two batches (Batch 5.1 and Batch 5.2) of aluminum-coated COP substrates having silicon dioxide second layers of varying thickness (2 samples per silicon dioxide layer thickness) and either reacted with GPTMS or not were evaluated.

In each batch, the aluminum first layer was 100 nm thick, with the silicon dioxide second layer thickness as follows in each of Batch 5.1 and Batch 5.2:

Batch 5.1: silicon dioxide layer thicknesses of 20 nm, 30 nm, 40 nm, 50 nm, 60 nm, 70 nm, 80 nm and 90 nm.

Batch 5.2: silicon dioxide layer thicknesses of 25 nm, 40 nm, 50 nm, 60 nm, 70 nm, 80 nm and 90 nm.

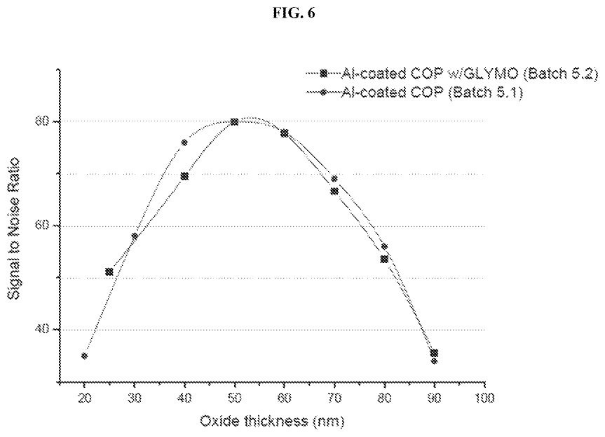

In Batch 5.2, the samples were additionally functionalized with a GPTMS layer. As can be seen in FIG. 6, the same optical performance was achieved across both batches. Hence, the GPTMS layer did not affect the overall optical performance.

### Example 6

COP substrates having bilayer coatings were compared to evaluate the effect of (1) selecting either a 200 nm thick silicon first layer and a 100 nm thick aluminum first layer, and (2) selecting a thickness of a silicon dioxide second layer. GNPs having a diameter of 100 nm were used. GNP scattering signals were obtained for two samples for each of the following batches:

Batch 6.1: a 200 nm thick silicon first layer and a second layer of silicon dioxide which is either 20 nm, 30 nm, 40 nm, 50 nm, 60 nm, 70 nm, 80 nm or 90 nm.

Batch 6.2: a 100 nm thick aluminum first layer and a second layer of silicon dioxide which is either 20 nm, 30 nm, 40 nm, 50 nm, 60 nm, 70 nm, 80 nm or 90 nm.

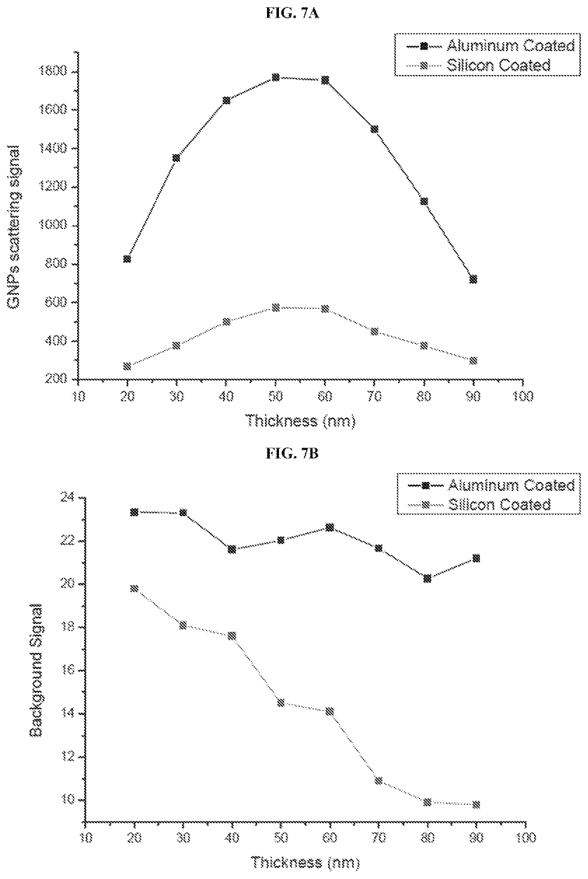

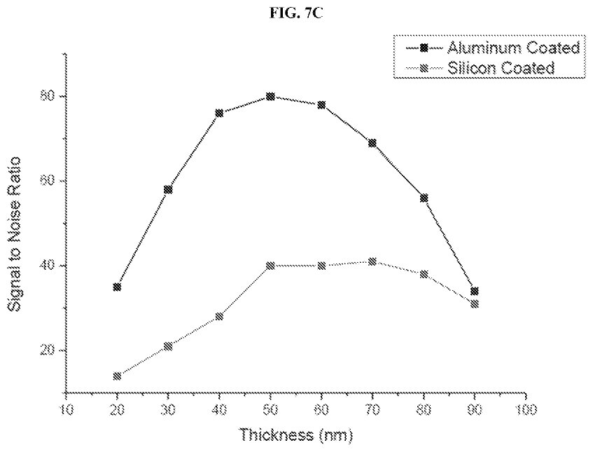

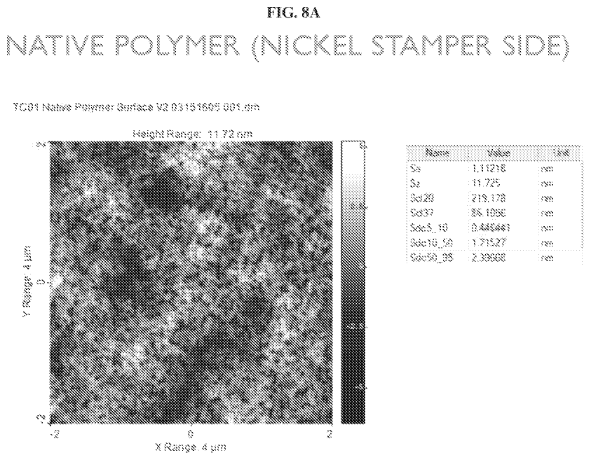

As shown in FIG. 7A, it was found that including a silicon dioxide layer which is between about 50 nm and about 60 nm optimizes GNP scattering signal. Additionally, it was found that the ratio of the Batch 6.1 (aluminum-coated) samples' GNP scattering signals to the Batch 6.2 (silicon-coated) samples' GNP scattering signals was approximately 3:1.

FIG. 7B shows the relationship between background signal and silicon dioxide layer thickness in each of the Batch 6.1 and Batch 6.2 samples. A low background signal was obtained for each of the tested substrates, and was found to decrease with increasing silicon dioxide layer thickness.

FIG. 7C shows the signal to noise ratio (SNR) for each of the Batch 6.1 and Batch 6.2 samples. For aluminum-coated substrates, the SNR was found to be optimized for 100 nm GNPs by selecting a silicon dioxide thickness of between about 40 nm and about 60 nm. For the silicon-coated substrates, the SNR was found to be optimized by selecting a silicon dioxide thickness of between about 50 nm and about 80 nm.

Hence, for 100 nm GNPs, aluminum-coated and silicon-coated COP substrates analyzed in Example 6 presented a maximum of the GNPs scattering signal when the layer thickness is around 50 nm. With current deposition methods, the silicon dioxide layer may be accurately and reproducibly deposited to allow for ease of industrialization.

### Example 7

The effect of surface roughness on signal and background noise was evaluated. Roughness was measured using atomic force microscopy (AFM).

It was found that significantly different roughness measurements were obtained via AFM on opposite sides of the same polymeric substrate sample.

COP substrates tested were prepared using a polished steel mold with high-quality nickel insert. The nickel insert was prepared as a 1:1 copy of a silicon wafer.

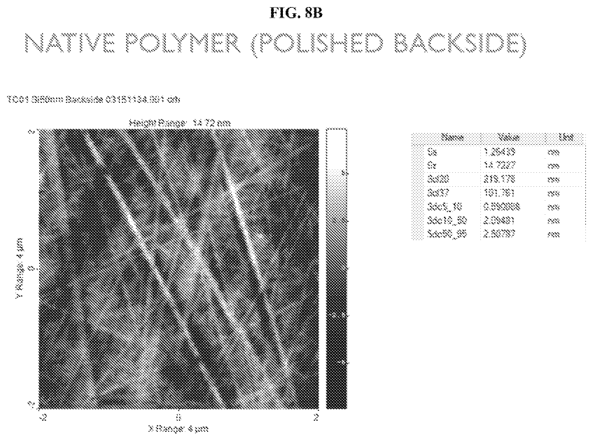

FIG. 8A shows the roughness of native (i.e., uncoated) polymer (COP) substrate on the mold with nickel insert side of the substrate.

FIG. 8B shows that the native COP substrate on the polished backside (i.e., the side opposite the nickel stamper side) was substantially rougher as compared to the mold with nickel insert.

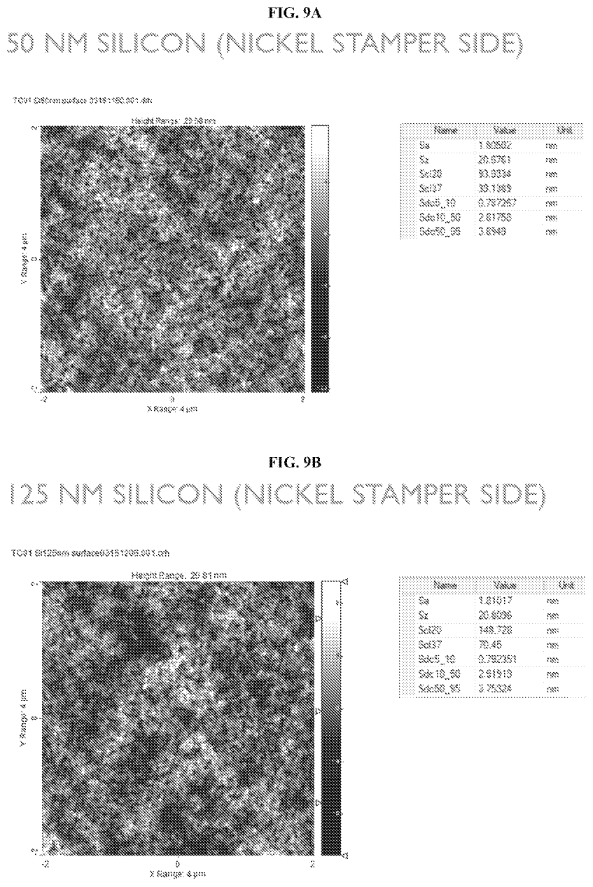

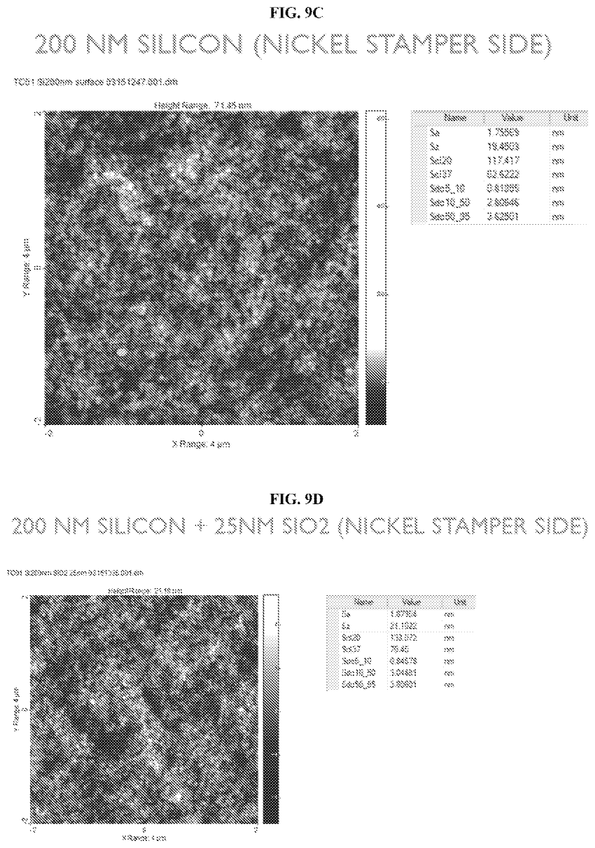

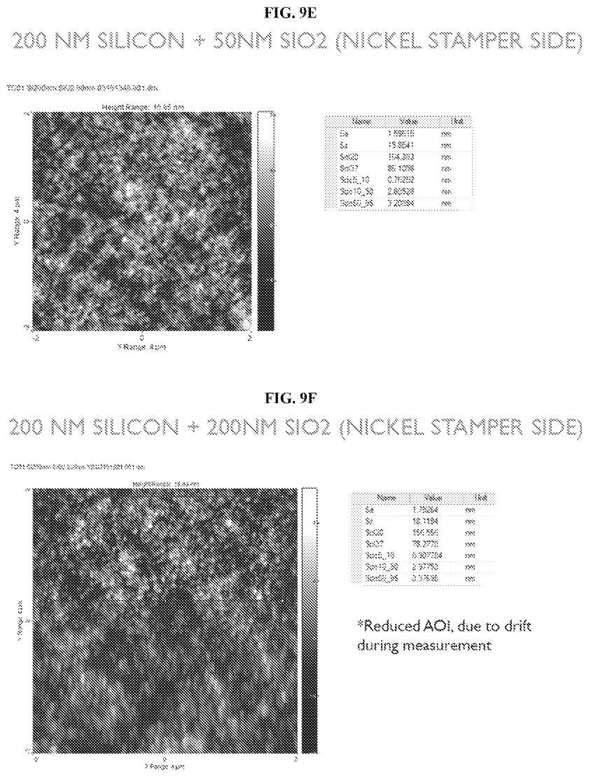

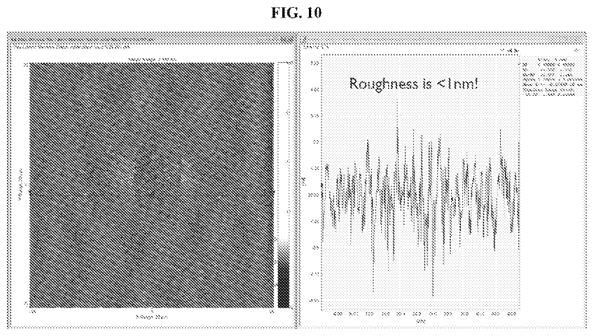

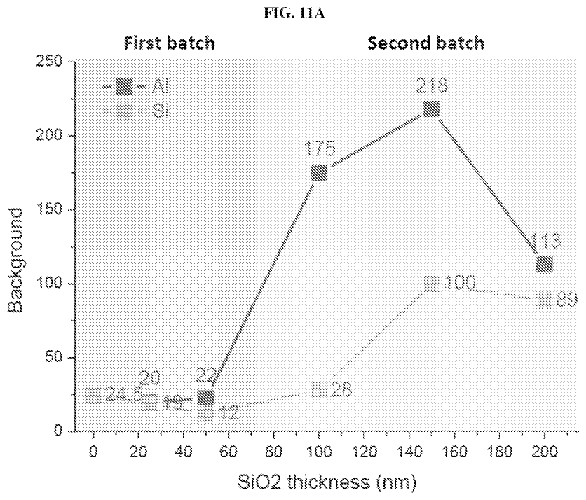

Similarly, FIGS. 9A, 9B, 9C, 9D, 9E and 9F, respectively, show the AFM measurements obtained for the nickel stamper sides of a COP substrate coated with 50 nm silicon (FIG. 9A), a COP substrate coated with 125 nm silicon (FIG. 9B), a COP substrate coated with 200 nm silicon (FIG. 9C), a COP substrate coated with 200 nm silicon+25 nm silicon dioxide (FIG. 9D), a COP substrate coated with 200 nm silicon+50 nm silicon dioxide (FIG. 9E), a COP substrate coated with 200 nm silicon+200 nm silicon dioxide (FIG. 9F)

It was thus found that a significant difference in surface finish (roughness) was obtained between the nickel stamper surface and the polished backside of a COP substrate, as well as surface quality, i.e., a lack of scratches on the nickel side

Additionally, it was found that there was no substantial influence of silicon sputter coating or silicon dioxide sputter coating.

### Example 8

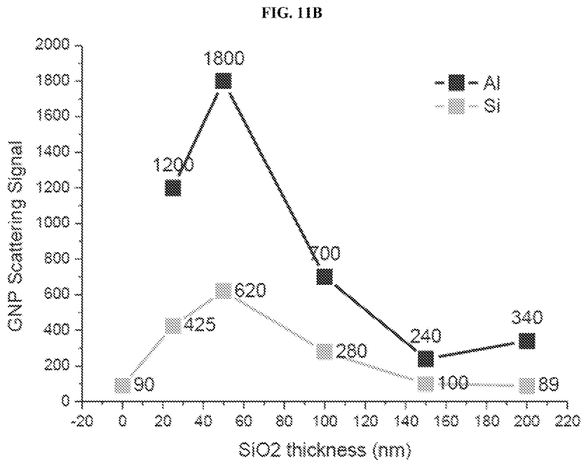

In a further surface roughness study using AFM, the surface finish (roughness) of COP substrates was compared with comparative reference substrates made of silicon, specifically silicon wafers having a roughness of less than 1 nm. The surface roughness measurement of the reference silicon wafer are shown in FIG. 10.

Table 1 below shows the roughness (“Roughness”) in nm for an exemplary COP substrate and the roughness of a reference silicon wafer (“Comparative Silicon Wafer”) in nm.

TABLE 1

| | | | | Comparative |
|---|---|---|---|---|
| | | | Difference | Silicon |
| Name | Unit | Roughness | % | Wafer |
| Ra | nm | 2.1771 | 90.61% | 0.2044 |
| Rq | nm | 2.7242 | 90.20% | 0.2671 |
| Rsk | | 0.0435 | 1933.50% | −0.7975 |
| Rku | | 3.0889 | −37.19% | 4.2377 |
| Ry | nm | 1.7033 | −6.18% | 1.8087 |
| Rt | nm | 1.7033 | −6.18% | 1.8087 |
| Rz | nm | 1.7033 | −6.18% | 1.8087 |
| R10z | nm | 1.4083 | −3.91% | 1.4633 |
| Rz_tph | nm | 1.4083 | −3.91% | 1.4633 |
| Rds | 1/μm | 0.4430 | −0.25% | 0.4441 |
| Rsc | 1/nm | 0.0010 | −42437.49% | 0.4207 |
| Rv | nm | 9.7311 | 88.53% | 1.1162 |
| Rp | nm | 0.7302 | 5.17% | 0.6925 |
| Rmean | nm | 0.00034625 | 97.50% | 0.00000867 |
| Rbi | | 0.5540 | −31.40% | 0.7279 |
| Rci | | 0.1759 | −633.65% | 1.2906 |
| Rvi | | 0.1179 | −42.15% | 0.1676 |
| Rpk | nm | 3.6072 | 92.45% | 0.2723 |
| Rk | nm | 6.8262 | 91.39% | 0.5875 |
| Rvk | nm | 2.7318 | 84.79% | 0.4156 |
| Rdc0_5 | nm | 2.3847 | 86.35% | 0.3256 |
| Rdc5_10 | nm | 1.0220 | 92.92% | 0.0723 |
| Rdc10_50 | nm | 4.0880 | 92.92% | 0.2894 |
| Rdc50_95 | nm | 4.4287 | 87.75% | 0.5426 |

### Example 9

COP substrates which were coated differently across two batches were analyzed.

In a first batch, COP substrates were coated with either (a) 200 nm silicon, (b) 200 nm silicon and 25 nm silicon dioxide, (c) 200 nm silicon and 50 nm silicon dioxide, (d) 100 nm aluminum and 25 nm silicon dioxide, or (e) 100 nm aluminum and 50 nm silicon dioxide.

In a second batch, COP substrates were coated with either (a) 100 nm aluminum and 100 nm silicon dioxide, (b) 100 nm aluminum and 150 nm silicon dioxide, (c) 100 nm aluminum and 200 nm silicon dioxide, (d) 200 nm silicon and 100 nm silicon dioxide, (e) 200 nm silicon and 150 nm silicon dioxide, or (f) 200 nm silicon and 200 nm silicon dioxide.

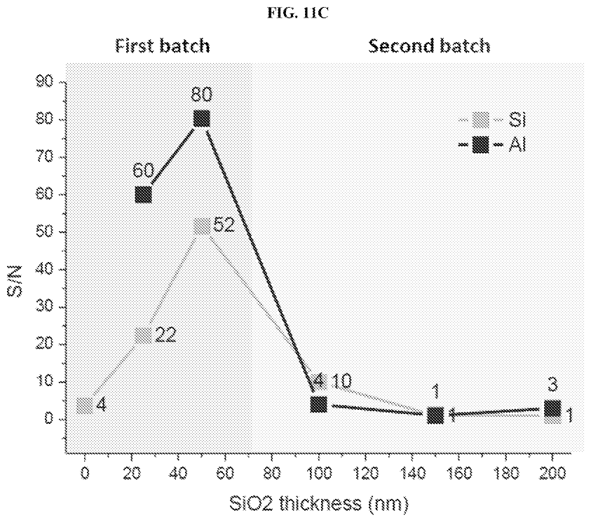

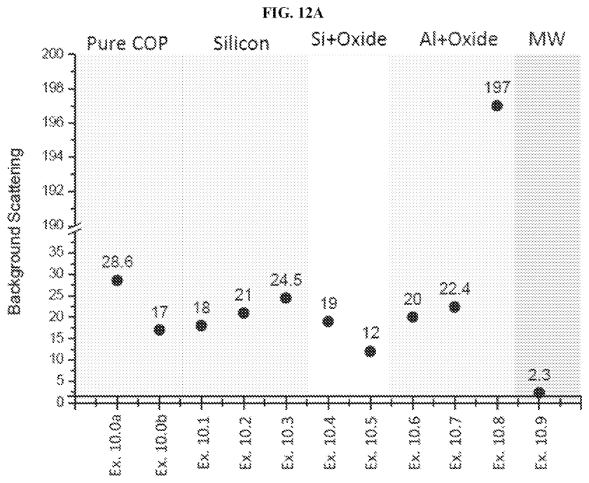

It was found that background scattering increased almost an entire order of magnitude in the second batch having increased silicon dioxide thicknesses. See FIG. 11A.

It was also found that GNP scattering signal obtained using the coated COP composites of the first and second batches (i.e., aluminum-coated and silicon-coated as a first layer) peaked for samples having silicon dioxide thicknesses of 50 nm. See FIG. 11B.

Additionally, it was found that the signal to noise ratio (SNR) peaked for the samples having 50 nm silicon dioxide using 100 nm GNPs for detection. See FIG. 11C.

It can thus be concluded that silicon dioxide layers in composites, in some embodiments, should be between about 25 nm and about 100 nm thick.

### Example 10

Additionally, various embodiments of the composite were prepared and assessed, as described in Table 2.

TABLE 2

| | | | | Coated |
|---|---|---|---|---|
| | Substrate | | | disc |
| Example | material | Layer 1 | Layer 2 | side |
| 10.0a | COP | None | None | n/a |
| 10.0b | COP | None | None | n/a |
| | (thin) | | | |
| 10.1 | COP | Silicon (50 nm) | None | Front |
| 10.2 | COP | Silicon (125 nm) | None | Front |
| 10.3 | COP | Silicon (200 nm) | None | Front |
| 10.4 | COP | Silicon (200 nm) | Silicon dioxide (25 nm) | Front |
| 10.5 | COP | Silicon (200 nm) | Silicon dioxide (50 nm) | Front |
| 10.6 | COP | Aluminum (100 nm) | Silicon dioxide (25 nm) | Front |
| 10.7 | COP | Aluminum (100 nm) | Silicon dioxide (50 nm) | Front |
| 10.8 | COP | Aluminum (100 nm) | Silicon dioxide (25 nm) | Back |
| 10.9 | Silicon | None | None | n/a |
| | (com- | | | |
| | parative | | | |
| | reference | | | |
| | disc) | | | |

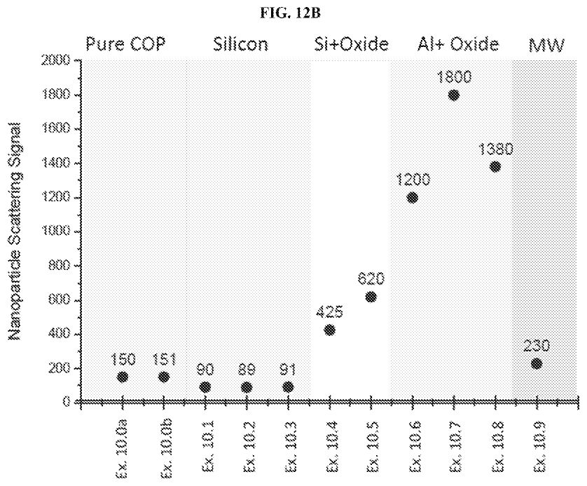

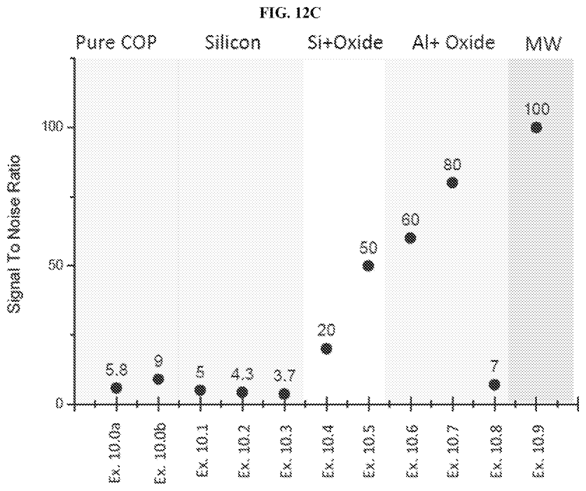

It was found that each coated substrate produced very low background scattering, except for the back-coated aluminum-based composite (Ex. 10.8), which presented a large background scattering increase. Thinner COP discs (Ex. 10.0b) of about 0.6 mm thickness presented a lower background scattering as compared to the other COP discs, which were 1.0 mm thick. See FIG. 12A.

It was also found that aluminum and silicon dioxide coated COP discs produced increased scattering relative to silicon and silicon dioxide coated COP discs and the silicon reference (Ex. 10.9). Additionally, it was found that substrates having thicker silicon oxide layers performed better than substrates having thinner oxide layers. Scattering was shown to be reduced in silicon-coated substrates (Exs. 10.1-10.3), about 60% of the scattering produced by COP blanks (Ex. 10.0a and 10.0b) and about 40% of the scattering produced by the silicon blank reference (Ex. 10.9). See FIG. 12B.

Additionally, it was found that the signal to noise ratio (SNR) for aluminum and silicon oxide coated substrates (Exs. 10.6-10.8) was comparable to the silicon reference blank (Ex. 10.9). Silicon and silicon dioxide coated substrates, particularly Ex. 10.5, showed good performance as well. Thinner COP discs (i.e., Ex. 10.0b) of 0.6 mm thickness performed better than thicker COP substrates (i.e., Ex. 10.0a) of 1.0 mm thickness. See FIG. 12C.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed:

1. A device, comprising:
a synthetic polymeric substrate having an upper surface;
a coating on at least a portion of the upper surface, wherein the coating comprises a first layer comprised of a material that reflects electromagnetic radiation and a second layer comprised of a material that is a dielectric and is transparent.

2. The device of claim 1, wherein the first coating layer is selected from the group consisting of aluminum, silver, gold, chromium, silicon, and dielectric materials.

3. The device of claim 1, wherein the first coating layer has a thickness of between about 10-1000 nm or between 10-500 nm or between 10-250 nm or between 10-200 nm or between 50-150 nm or between 75-125 nm.

4. The device of claim 1, wherein the second coating layer is a metalloid, an oxide of a metalloid, or a synthetic polymer.

5. The device of claim 4, wherein the metalloid or metalloid oxide is boron, silicon, germanium, arsenic, antimony, tellurium, polonium, or an oxide thereof.

6. The device of claim 1, wherein the second layer has a thickness of between about 10-500 nm or between 20-200 nm or between 20-100 nm or between 50-100 nm or between 75-100 nm or between 70-90 nm.

7. The device of claim 1, wherein the second layer comprises silicon dioxide.

8. The device of claim 1, wherein the first layer is at least as thick as the second layer.

9. The device of claim 8, wherein the first layer is aluminum and the second layer is silicon dioxide.

10. The device of claim 9, wherein the first layer and the second layer have a thickness within about 30%, 25%, 20%, 15%, or 10% of each other.

11. The device of claim 1, wherein an overcoating is applied with 3-glycidyloxypropyl)trimethoxysilane (GPTMS), 3-aminopropyltrimethoxysilane (APTMS), or 3-aminopropyltriethoxysilane (APTES).

12. The device of claim 1, wherein the substrate has a surface roughness before or after coating, essentially equivalent to a silicon wafer suitable for semiconductor production.

13. The device of claim 1, wherein the substrate has a thickness of less than 1 mm, less than 0.5 mm, less than 0.3 mm, less than 0.25 mm, less than 0.2 mm, less than 0.1 mm.

14. The device of claim 1, wherein the substrate consists of cyclic olefin copolymer or a cyclic olefin polymer.

15. The device of claim 1, wherein the substrate comprises a thermoplastic material.

16. The device of claim 1, wherein the upper surface of the substrate has a flatness of less than about 100 μm.

17. A device, comprising:
a composite, solid support member comprised a synthetic polymeric substrate with an upper surface and a lower surface, the upper surface comprising at least a bilayer coating comprised of a reflective layer deposited on the upper surface and a dielectric, transparent layer deposited on the reflective layer; and
a plurality of binding members immobilized to the composite, solid support member.

18. The device of claim 17, wherein the plurality of binding members comprises a first binding member for a first analyte and a second binding member for a second analyte.

19. The device of claim 17, wherein the plurality of binding members comprises a haloalkane dehalogenase binding tag.

20. The device of claim 17, wherein the plurality of binding members comprises a protein, an antibody or a peptide.

21. The device of claim 17, wherein the plurality of binding members comprises a ligand with specific binding for a binding tag that is part of a fusion protein comprising an antibody or antibody fragment that binds an analyte of interest.

22. The device of claim 21, wherein the binding tag is a haloalkane dehalogenase or a biotin ligase binding site.

23. The device of claim 21, wherein the ligand is a synthetic organic compound.

24. The device of claim 23, wherein the compound comprises a chloroalkane linker.

25. The device of claim 21, wherein the ligand is biotin.

26. A kit for detecting a biological analyte of interest in a test sample, comprising:
an assay comprising a detection zone, the detection zone comprising the device of claim 1 and a plurality of binding members immobilized to the device;
a container comprising a population of detectable particles; and
instructions for use.

27. The kit according to claim 26, wherein the detectable particles comprise a magnetic core and a shell, wherein the shell consists essentially of a metal selected from the group consisting of gold, silver, platinum, palladium, iridium, osmium, rhodium, ruthenium.

28. The kit according to claim 26, wherein the detectable particles consist essentially of a metal selected from the group consisting of gold, silver, platinum, palladium, iridium, osmium, rhodium, ruthenium, or wherein the detectable particles do not have a core-shell structure.

29. The kit according to claim 26, wherein the detectable particles have an average diameter ranging from about 1 nm to about 1500 nm, or from about 25 nm to about 500 nm, or from about 50 nm to about 250 nm, or from 100-180 nm.

30. The kit according to claim 27, wherein the magnetic core has a diameter ranging from about 5 nm to about 150 nm and the shell has a thickness ranging from about 10 nm to about 50 nm.

31. The kit according to claim 27, wherein the detectable particles further comprise an intermediate layer provided between the core and the shell.

32. A method for the detection of a biological analyte in a fluid sample, comprising:
contacting a device according to claim 1 with a fluid sample suspected of comprising the biological analyte of interest and with a detectable particle associated with a binding species for the analyte of interest, where the device comprises an immobilized member with binding for the binding species; and
analyzing the device with an optical instrument for presence or absence of the detectable particle.
